(19) United States
(12) Patent Application Publication
(10) Pub. No.: US 2007/0231291 A1
(43) Pub. Date: Oct. 4, 2007

(54) Polymeric anti-microbial agents

(76) Inventors: Xinyu Huang, Hillsborough, NJ (US); Ted Deisenroth, Brookfield, CT (US); Andrea Preuss, Basel (CH); Sophie Marquais-Bienewald, Hegenheim (FR); Carmen Hendricks-Guy, White Plains, NY (US); John Jennings, Moycullen (IE)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 11/656,863

(22) Filed: Jan. 23, 2007

Related U.S. Application Data
(60) Provisional application No. 60/762,808, filed on Jan. 27, 2006.

(51) Int. Cl. A61K 31/787; C08L 63/00

(52) U.S. Cl. 424/78.3; 525/438

(57) ABSTRACT

Polymeric anti-microbial agents produced by substituting the nitrogen atoms in the backbone of ethylenimine polymers are provided. The agents are believed to have low human toxicity while being effective against a variety of pathogens and are useful in applications involving human contact, such as cosmetics, hair care products and textiles, as well as in applications with much less human contact, such as coatings.

This application claims benefit under 35 USC 119(e) of U.S. provisional application No. 60/762,808, filed Jan. 27, 2006, incorporated herein in its entirety by reference.

Polymeric anti-microbial agents derived from polyethylenimine are provided. The agents are believed to have low human toxicity while being effective against a variety of pathogens and are useful in applications involving human contact, such as cosmetics, hair care products and textiles, as well as in applications with much less human contact, such as coatings.

Anti-microbial compounds are widely used and accepted as part of numerous products and materials. Anti-bacterial soaps, anti-fungal treatments for plants, topical medical treatments, anti-fouling coatings and disinfecting cleaners are just a few common uses of anti-microbial materials.

An apparent dilemma in the use of anti-microbial compounds is that such compounds must be active against living organisms but not be toxic toward humans, animals or desirable plants. Disclosed herein are compounds effective against a variety of harmful microbes expected to be less harmful to humans than many other anti-microbial compounds due in part to the polymeric nature of the compounds of the present invention.

U.S. Pat. Nos. 6,090,772; 5,955,408; 6,071,866; 6,358,906, incorporated herein in their entirety by reference, and WO96/06152 disclose compositions useful in personal applications comprising triclosan as an anti-bacterial agent.

U.S. Pat. No. 5,635,462, incorporated herein in its entirety by reference, also discloses compositions comprising an anti-bacterial agent.

WO98/55096 discloses antimicrobial wipes having a porous sheet impregnated with an antibacterial composition containing an active antimicrobial agent.

U.S. Pat. No. 6,861,397, incorporated herein in its entirety by reference, discloses personal care and cleaning compositions having enhanced deposition of a topically active compound including antibacterial agents.

U.S. Pat. No. 6,872,241, incorporated herein in its entirety by reference, discloses anti-pathogenic air filtration media and air handling devices having protective capabilities against infectious airborne microorganisms.

U.S. Pat. No. 3,116,969, incorporated herein in its entirety by reference, describes a filter having an alkyl aryl quaternary ammonium chloride antiseptic compound that is held onto the filter fibers by a tacky composition that includes a hygroscopic agent, a thickening agent and a film forming agent.

According to its English language abstract, International Publication No. WO 00/64264 discloses a bactericidal organic polymeric material for filters made of a polymer base comprising a backbone and bonded thereto a polymeric pendant group comprising units derived from an N-alkyl-N-vinylalkylamide and triiodide ions.

U.S. Pat. No. 5,405,919, incorporated herein in its entirety by reference, discloses methods for bonding or coupling biologically active diazeniumdiolate NO-releasing groups to polymers including polyolefins, polyethylenimine, polyesters, polyethers, polyurethanes and the like.

JP-09157113-A, JP 09012717-A and JP 07188698 disclose the use of alkyl (C8 to C30), acyl and hydroxyalkyl modified polyethylenimine as antimicrobial agents which are particularly effective with salts of Zn, Cu and Ag.

Klibanov, Biotechnol. Prog. 2002, 18, 1082-1086; Biotech. and Bioengin. 2003, 83, 168-172 describes N-alkylated polyethylenimine derivatives effective as antimicrobial agents.

It is important that anti-microbial compounds, for example, as such as those found in antibacterial compositions provide a substantial and broad spectrum reduction in microorganism populations quickly and without problems associated with toxicity and skin irritation. Antibacterial activity is assessed against a broad spectrum of microorganisms, including both Gram positive and Gram negative microorganisms. The log reduction, or alternatively the percent reduction, in bacterial populations provided by the antibacterial composition correlates to antibacterial activity. A log reduction of 3-5 is most preferred, a 1-3 reduction is preferred, whereas a log reduction of less than 1 is least preferred, for a particular contact time, generally ranging from 15 seconds to 5 minutes. Thus, a highly preferred antibacterial composition exhibits a 3-5 log reduction against a broad spectrum of microorganisms in a short contact time.

The state of art for antimicrobial solution is the cocktail method, which provides a broad spectrum of antimicrobial activity by mixing two or more antimicrobial compounds. This method is usually associated with compatibility issues because of the difference of the physical and chemical properties of antimicrobial compounds, for example, different stability, solubility and leaching rate. The advantage of antimicrobial polymers is a broad spectrum of antimicrobial activity can be achieved by combination of different functional groups onto the same polymer chain without generating any compatibility issues. Functional groups can also be introduced to tailor the physical and chemical properties of the antimicrobial polymers and therefore improve their performance in applications. It has been demonstrated by our experiments that, by further introducing functional groups onto the polymer chains, the solubility of the antimicrobial polymer in water and/or glycol can be significantly increased without any influence on the antimicrobial activities.

### SUMMARY OF THE INVENTION

The present invention provides new anti-microbial polyethylenimine compounds. These polymeric and oligomeric compounds are highly active against microbes upon contact, and remain active over a prolonged period of time due in part to their size and polymeric nature which makes them less susceptible to being unintentionally removed. The compounds are also expected to be less harmful upon human contact than other compounds that are more readily absorbed through the skin or made bio-available by dispersion into the environment.

### DESCRIPTION OF THE INVENTION

The present invention provides an antimicrobial ethylenimine polymer or co-polymer, wherein 10-100% of the nitrogen atoms (also referred to herein as N atoms) of the polymer or co-polymer backbone are substituted by one or more substituents a-d:
a) C₁₋₂₄ alkyl, C₃₋₂₄ alkenyl, C₁₋₂₄ alkylcarbonyl or C₃₋₂₄ alkenylcarbonyl which are uninterrupted or interrupted one or more times by one or more oxygen atoms, sulfur atoms, —SO— or —SO₂—, and which are substituted one or more times by one or more moieties C₃₋₆ cycloalkyl, —OR, —COOR, —COOM, —SO₃M, —SO₃H, phosphonic acid, halogen, —CONR′R, —NR′R, phosphonate salt, ammonium salt or group of the formulae 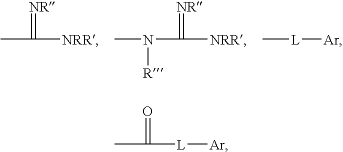
or a group —Si(G)₃ wherein each G is independently hydroxyl, C₁₋₄ alkyl or C₁₋₄ alkoxy,
with the proviso that uninterrupted C₁₋₂₄ alkyl is not substituted by biguanide, C₃₋₆ cycloalkyl, —COOM, —COOR where R is an unsubstituted alkyl, —OR where R is H or unsubstituted alkylcarbonyl or —CONR′R unless at least one other of the substituents is also present;
b) heterocycle of the formulae 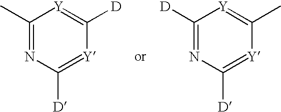
wherein Y and Y′ are independently N, C—R, C—OR or C—NRR′ and D and D′ are independently R, —OR or —NRR′;
c) group of the formulae 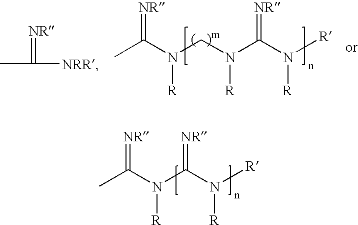
wherein m and n independently are 1, 2, 3, 4, 5 or 6;
or
d)-L-Poly where Poly is branched or unbranched polymer or oligomer selected from polyether, polysiloxane, styrenic polymer or polyol;
wherein
R, R′ and R″, independently of each other are hydrogen; a group -L-Ar, 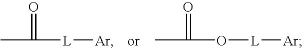
C₁₋₂₄ alkyl, C₃₋₂₄ alkenyl, C₃₋₆ cycloalkyl or C₁₋₂₄ alkylcarbonyl which are uninterrupted or interrupted one or more times by one or more oxygen atoms, sulfur atoms, carbonyl, —COO—, —CONH—, —NH—, —CON(C₁₋₈ alkyl)- or —N(C₁₋₈ alkyl)-, which uninterrupted or interrupted alkyl, alkenyl, cycloalkyl or alkylcarbonyl are unsubstituted or substituted one or more times by one or more halogen, —OH, C₇₋₁₂ aralkyl, C₂₋₁₂alkylcarbonyl, C₁₋₂₄alkoxy, C₂₋₂₄alkylcarboxy, —COOM, —CONH₂, —CON(H)(C₁₋₈alkyl), —CON(C₁₋₈ alkyl)₂, —NH₂, —N(H)(C₁₋₈ alkyl), —N(C₁₋₈ alkyl)₂, —SO₃M, phenyl, phenyl substituted one or more times by one or more C₁₋₈ alkyl, naphthyl, naphthyl substituted one or more times by one or more C₁₋₈ alkyl, purine, pyridine, pyrimidine, triazine or imidazole which purine, pyridine, pyrimidine, triazine or imidazole are unsubstituted or substituted by one or more C₁₋₁₂ alkyl wherein the purine, pyridine, pyrimidine, triazine or imidazole is neutral or ionically charged, amidine, guanidine, ammonium salt, phosphonic acid, phosphonate salt or a group 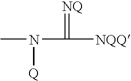
wherein each Q or Q′ is independently hydrogen, C₁₋₁₂alkyl, phenyl or benzyl;
or
when attached to a nitrogen atom, R and R′, together with the nitrogen atom to which they are attached, form a 5-, 6- or 7-membered ring which is uninterrupted or interrupted by —O—, —NH— or —N(C₁₋₁₂ alkyl)-;
L is a direct bond, C₁₋₁₂ alkylene which is uninterrupted or interrupted by one or more oxygen atoms and which is unsubstituted or substituted one or more times by one or more —OH, C₁₋₈ alkyl, C₁₋₂₄ alkoxy, C₂₋₂₄alkylcarboxy, —NH₂, —N(H)(C₁₋₈alkyl), —N(C₁₋₈ alkyl)₂ or ammonium salt:
Ar is C₆₋₁₀ aromatic or C₁₋₉ saturated or unsaturated heterocycle which are unsubstituted or substituted one or more times by one or more halogen, —OH, C₁₋₂₄ alkoxy, C₂₋₂₄ alkylcarboxy, —COOQ″, —CONH₂, —CON(H)(C₁₋₈ alkyl), —CON(C₁₋₈ alkyl)₂, —NH₂, —N(H)(C₁₋₈ alkyl), —N(C₁₋₈alkyl)₂, —SO₃M, SO₃H, ammonium salt, phosphonic acid, phosphonate salt, C₁₋₂₄ alkyl which is unsubstituted or substituted one or more times by one or more halogen, phenyl which is unsubstituted or substituted by one or more times by one or more C₁₋₈ alkyl, naphthyl, purine, pyridine, pyrimidine, triazine or imidazole which purine, pyridine, pyrimidine, triazine or imidazole are unsubstituted or substituted by one or more C₁₋₁₂ alkyl wherein the purine, pyridine, pyrimidine, triazine or imidazole is neutral or ionically charged; wherein Q″ is hydrogen, metal cation, glycol ether, polysiloxane, phenyl or benzyl, or phenyl or benzyl substituted one or more times by one or more halogen, hydroxy, C₁₋₂₄ alkoxy or C₁₋₁₂ alkyl,
M is a metal cation or an ammonium cation;
and when the N atom of the ethylenimine polymer is tetra substituted, it is a cation with a corresponding counter anion.

For example the present invention provides an antimicrobial ethylenimine polymer or co-polymer as described above wherein at least a portion of the substituents are C₁₋₂₄ alkyl, C₃₋₂₄ alkenyl, C₁₋₂₄ alkylcarbonyl or C₃₋₂₄ alkenylcarbonyl which are uninterrupted or interrupted one or more times by one or more oxygen atoms, sulfur atoms, —SO— or —SO₂—, and which are substituted one or more times by one or more —OR, —COOR, —COOM, —NR′R, —SO₃M, —SO₃H, halogen, —NR′R, ammonium salt or group of the formulae 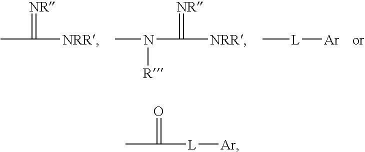
with the proviso that uninterrupted C₁₋₂₄ alkyl is not substituted by biguanide, —COOM, —COOR where R is an unsubstituted alkyl, or —OR where R is H or unsubstituted alkylcarbonyl, unless at least one other of the substituents is also present;
for example, an antimicrobial ethylenimine polymer or co-polymer wherein at least a portion of the substituents are C₁₋₂₄ alkyl or C₁₋₂₄ alkylcarbonyl substituted by one or more halogen, ammonium salt, 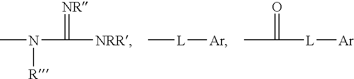
or —OR wherein R is -L-Ar, or 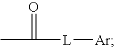
for example an antimicrobial ethylenimine polymer or co-polymer wherein at least a portion of the substituents are C₁₋₂₄ alkyl substituted one or more times by one or more halogen or ammonium salt, C₁₋₂₄ alkylcarbonyl substituted one or more times by one or more halogen or ammonium salt or at least a portion of the substituents are benzyl, benzoyl or benzyl or benzoyl substituted one or more times by one or more halogens, hydroxyl, C₁₋₁₂ alkyl, C₁₋₁₂ alkoxy or C₁₋₁₂ alkylcarboxy;
for example an antimicrobial ethylenimine polymer or co-polymer, wherein at least a portion of the substituents are C₁₋₂₄ alkyl or C₁₋₂₄ alkylcarbonyl substituted by at least one group selected from ammonium salt, phenoxy, benzyloxy, substituted phenoxy, substituted benzyloxy, benzyl, substituted benzyl, 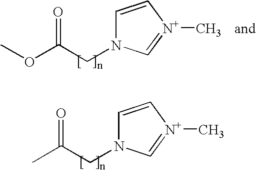
where n is a number from 1 to 12;
for example an antimicrobial ethylenimine polymer or co-polymer, wherein at least a portion of the 10-100% of the N atoms of the polymer or co-polymer backbone which are substituted are substituted by one or more groups 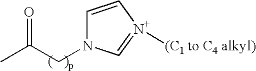
wherein p is 1, 2, 3, 4, 5, or 6.

For example, at least a portion of the 10-100% of the N atoms of the antimicrobial ethylenimine polymer or co-polymer backbone which are substituted are substituted by one or more C₁₋₂₄ alkyl substituted by at least one group OR and at least one halogen, NR′R, SO₃M, SO₃H, ammonium salt or a group of the formulae 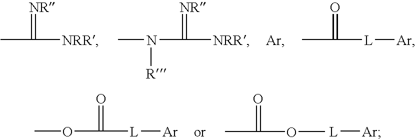
for example, C₁₋₂₄ alkyl substituted by at least one group OR and at least one halogen, NR′R, ammonium salt or a group of the formulae 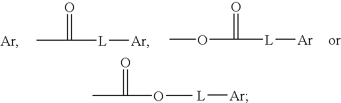
for example, C₁₋₂₄ alkyl substituted by OH and a group selected from ammonium salt, benzyl, substituted benzyl, and

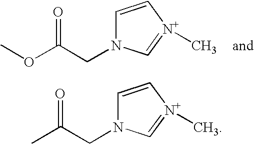

For example, the present invention provides an antimicrobial ethylenimine polymer or co-polymer wherein at least a portion of the substituents on the 10-100% of the N atoms of the polymer or co-polymer backbone which are substituted are selected from the group consisting of C₂₋₂₄ alkyl, C₂₋₂₄ alkylcarbonyl, C₃₋₂₄ alkenyl, and C₃₋₂₄ alkenylcarbonyl interrupted one or more times by one or more oxygen atoms, sulfur atoms, —SO— or —SO₂—, which are unsubstituted or substituted one or more times by one or more halogen, —OR, —COOR, —COOM, —CONR′R, —NR′R, —SO₃M, —SO₃H, phosphonic acid, phosphonate salt, ammonium salt or a group of the formulae 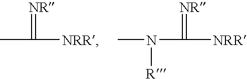
or -L-Ar;
for example said interrupted alkyl or alkylcarbonyl, unsubstituted or substituted one or more times by one or more halogen, —OR, —COOR, —COOM, —SO₃M, —SO₃H, ammonium salt or a group of the formulae 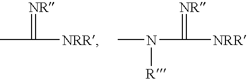
or -L-Ar;
for example said interrupted alkyl or alkylcarbonyl, substituted one or more times by one or more halogen, —OR, —COOR, —COOM, —SO₃M, —SO₃H, ammonium salt or a group of the formulae 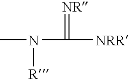
or -L-Ar.

For example, the present invention provides an antimicrobial ethylenimine polymer or co-polymer, wherein at least a portion of the 10-100% of the N atoms of the polymer or co-polymer backbone which are substituted are substituted by one or more 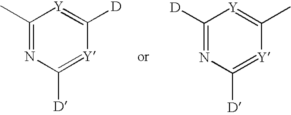
wherein Y and Y′ are independently N, C—R, C—OR or C—NRR′ and
D and D′ are independently R, OR or NRR′;
for example, an antimicrobial ethylenimine polymer or co-polymer wherein at least a portion of the substituents are 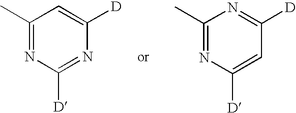
wherein D and D′ are independently R, OR or NRR′ wherein R and R′ are independently hydrogen, ammonium salt, C₁₋₂₄ alkyl, C₁₋₂₄ alkanoyl which are unsubstituted or substituted one or more times by one or more halogen, hydroxyl or ammonium salt; or R and R′ are independently -L-Ar, 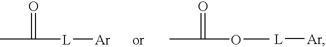
wherein L is a direct bond or C₁₋₁₂ alkylene and
Ar is phenyl or phenyl substituted one or more times by one or more halogen, —OH, C₁₋₂₄ alkoxy, C₂₋₂₄alkylcarboxy, —COOH, —COOM, —CONH₂, —CON(H)(C₁₋₁₂alkyl), —CON(C₁₋₁₂ alkyl)₂, —NH₂, —N(H)(C₁₋₁₂ alkyl), —N(C₁₋₁₂ alkyl)₂, ammonium salt, C₁₋₁₂ alkyl or alkyl substituted one or more times by one or more halogen.

For example, at least a portion of the 10-100% of the N atoms of the antimicrobial ethylenimine polymer or co-polymer backbone which are substituted are substituted by a group -L-Poly where Poly is a polymer or oligomer selected from polyether and polysiloxane;
for example, at least a portion of the substituents on the nitrogen atoms of the polymer or co-polymer backbone are 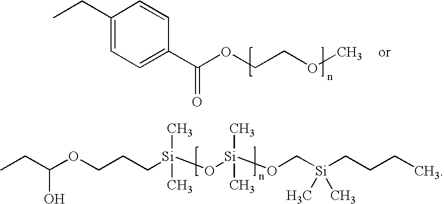
C₁₋₉ saturated or unsaturated heterocycle is a monocylclic or polycyclic ring of at least 3 atoms, containing 1-9 carbon atoms which heterocycle may also be ionically charged.

For example, C₁₋₉ saturated or unsaturated heterocycle is a 5, 6, or 7 membered ring containing 1, 2 or 3 nitrogen atoms which may be fused to another carbocylic or heterocyclic ring;

for example, C₁₋₉ saturated or unsaturated heterocycle is a 5, 6, or 7 membered ring containing 1, 2 or 3 nitrogen atoms which may be fused to a benzene ring;

for example, C₁₋₉ saturated or unsaturated heterocycle is a purine, imidazole, pyridine, pyramidine or triazole ring;

wherein the heterocyle may be substituted as described above and which heterocycle may also be ionically charged.

Alkyl is a straight or branched chain of the specified number of carbon atoms and is for example methyl, ethyl, n-propyl, n-butyl, sec-butyl, tert-butyl, n-hexyl, n-octyl, 2-ethylhexyl, n-nonyl, n-decyl, n-undecyl, n-dodecyl, n-tridecyl, n-tetradecyl, n-hexadecyl, n-octadecyl or docosanyl and the like.

Alkenyl is a straight or branched chain of the specified number of carbon atoms containing one or more carbon-carbon double bonds and is for example n-propenyl, n-butenyl, sec-butenyl, n-hexenyl, n-octenyl, n-hexadienyl, n-octadienyl, 2-ethylhexenyl, n-nonenyl, n-decenyl, n-undecenyl, n-dodecenyl, n-tridecenyl, n-tetradecenyl, n-hexadecenyl, n-octadecenyl, n-dodecadienyl, n-tetradecadienyl, n-hexadecadienyl, n-hexadecatrienyl, n-octadecadienyl, n-octadecatrienyl.

Alkanoyl is a straight or branched chain of the specified number of carbon atoms which has a carbonyl at the point of attachment.

An ammonium salt is, for example, unsubstituted ammonium, ammonium substituted 1, 2 or 3 times by one or more groups selected from

C₆₋₁₀aryl, C₁₋₂₄alkyl, C₁₋₂₄branched alkyl, C₁₋₂₄alkyl and branched alkyl interrupted by one or more oxygen atoms, carbonyl, carboxy or C₆₋₁₀ arylene,

and said aryl, alkyl, branched alkyl, interrupted alkyl and interrupted branched alkyl substituted by alkyl, aryl, OH, OAlkyl, OAcyl; plus a corresponding counter anion.

The ammonium salt may also comprise a ring or polycycle, which ring or polycycle may be substituted.

For example, the ammonium salt is tris benzyl ammonium or mono-, di-, or tri-C₁₋₂₄alkylammonium wherein each alkyl group can be the same or different, mono-, di-, or tri-benzyl, mono-, di-, or tri-C₁₋₂₄hydroxyalkylammonium wherein each alkyl group can be the same or different.

For example, the ammonium salt is di- or tri-substituted ammonium wherein each of the substituents are independently chosen from C₁₋₂₄alkyl, benzyl and C₁₋₂₄hydroxyalkyl.

The C₁₋₂₄alkyl, benzyl and C₁₋₂₄hydroxyalkyl groups of the substituted ammonium salts, may also be substituted by one or more C₁₋₈alkyl or branched alkyl, hydroxy, C₁₋₂₄carboxy ester, C₁₋₂₄alkyloxy, C₁₋₂₄acyloxy or halogen.

When M is an ammonium cation, it is for example, unsubstituted ammonium, ammonium substituted 1, 2, 3 or 4 times by one or more groups selected from C₁₋₂₄alkyl, C₁₋₂₄branched alkyl, said alkyl and branched alkyl interrupted by one or more oxygen atoms, C₆₋₁₀aryl, C₇₋₉ aralkyl, and said alkyl, branched alkyl, interrupted alkyl and interrupted branched alkyl, and aryl substituted by alkyl, OH, OC₁₋₂₄alkyl, OC₁₋₂₄acyl.

The N atoms of the ethylenimine polymer or co-polymer may be substituted by many of the different substituents described above, a few of the substituents described above or one of the substituents described above. A single substituent need not substitute 10% or more of the N atoms of the ethylenimine polymer or co-polymer backbone as long as other substituents described above, also referred to hereafter as inventive substituents, are also present so that at least 10% of the N atoms are substituted with one or more of the inventive substituents.

Examples of the inventive substituents include:

benzyl, benzoyl,

benzyl or benzoyl substituted by one or more halogens, C₁₋₈ alkyl, ammonium salts, hydroxy groups, acyloxy groups carboxy acid, acid salt and/or ester groups;

C₁₋₂₄ alkyl or alkyl carbonyl substituted by one or more ammonium salts, phenyl, naphthyl, substituted or unsubstituted heterocycle such as purine, pyridine, pyrimidine, pyrimidine or triazine;

C₁₋₂₄ alkyl or alkyl carbonyl substituted by one or more ammonium salts, halogens, groups OR, phenyl, naphthyl, substituted or unsubstituted heterocycle and also bearing at least one other different substituent selected from halogens, hydroxy groups, acyloxy groups, carboxy acid, acid salt and/or ester groups, such as an alkyl substituted by hydroxy and halogen, or acyl and substituted purine;

said alkyl interrupted by oxygen and substituted by benzyl, benzoyl, substituted benzyl, substituted benzoyl, substituted or unsubstituted heterocycle such as purine, pyridine, pyrimidine, pyrimidine, triazine, acyl group further substituted by said heterocycle or one or more halogens;

A heterocycle such as purine, pyramidine or triazine heterocycle unsubstituted or substituted by one or more C₁₋₈ alkyl, alkyl amine, aryl amine, phenyl, benzyl, substituted phenyl or benzyl, acyloxy groups, carboxy acid, acid salt and/or ester groups.

For example the inventive substituents are selected from the group consisting of benzyl substituted 1-5 times by F, Cl, Br or I or any combination of F, Cl, Br or I;
pyramidine or triazine of the following formulae 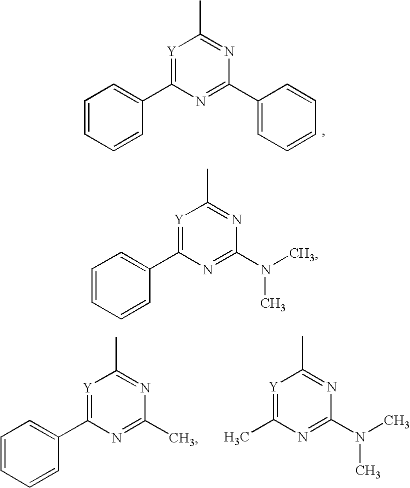
where Y is CR or N;
C₁₋₂₄ alkyl or alkyl carbonyl substituted by one or more 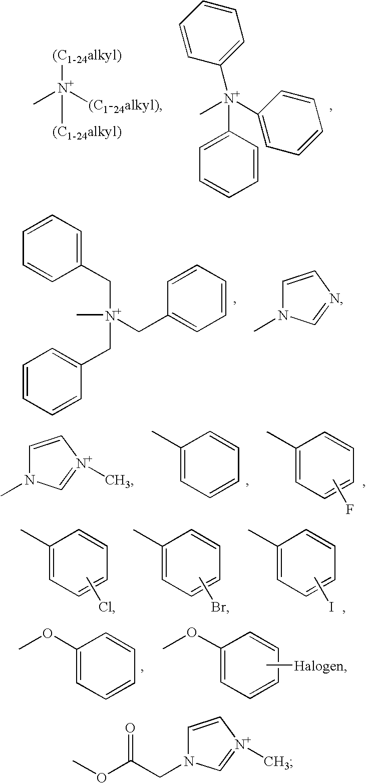
and C₁₋₂₄ alkyl or alkyl carbonyl substituted by one or more 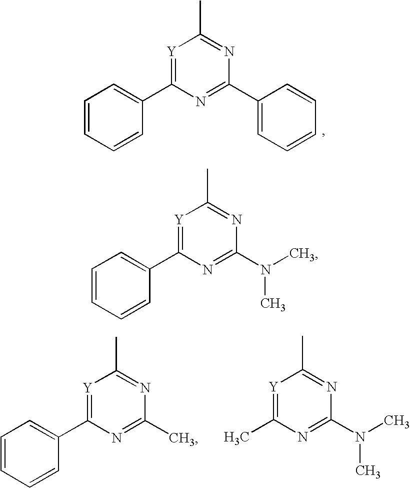
where Y is C or N.

For example, the inventive substituents include the following formulae, isomers of the following formulae and homologues of said formulae and homologues of said isomers: 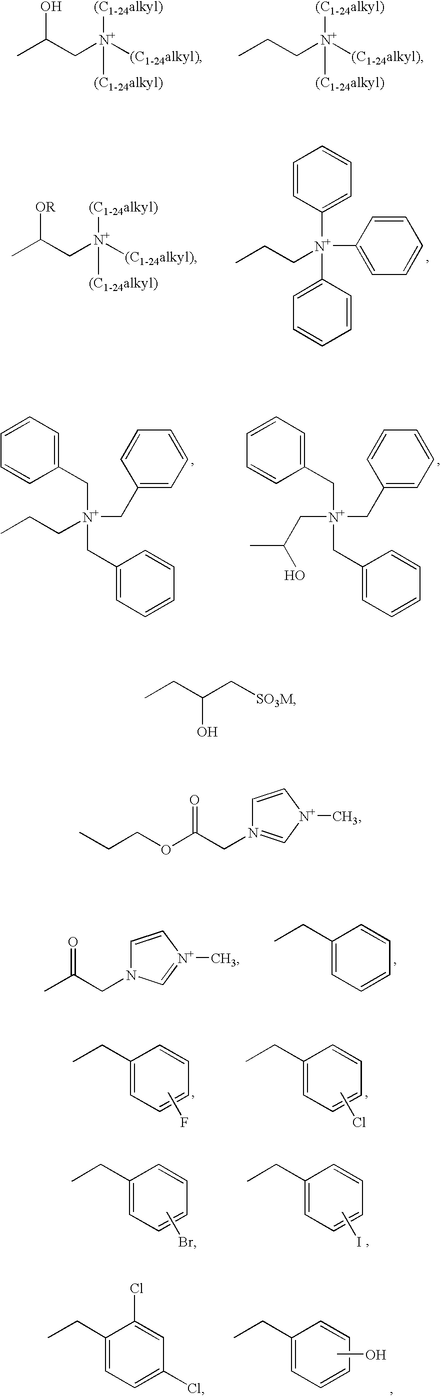 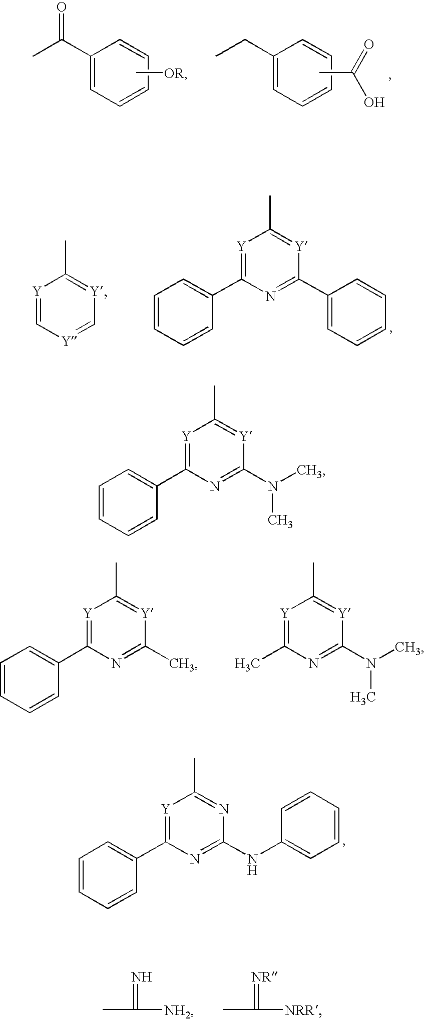
wherein Y, Y′ and Y″ are C or N and R is as defined above.

Remaining nitrogen atoms may be unsubstituted or substituted by C₁₋₂₄ alkyl or said alkyl substituted by —OR, COOR, COOM wherein R and M are as described above. The invention therefore includes ethylenimine polymers or copolymers wherein 10-100%, especially 10-99%, of the nitrogen atoms carry an inventive substituent, while the remaining nitrogen atoms, e.g. 1-90%, are unsubstituted, or are substituted by C₁₋₂₄ alkyl or said alkyl substituted by —OR, COOR, COOM wherein R and M are as described above, or a portion is unsubstituted while the rest is substituted by C₁₋₂₄ alkyl or said alkyl substituted by —OR, COOR, COOM wherein R and M are as described above.

In one embodiment of the invention, at least a portion of the 10-100% of the N atoms of the an antimicrobial ethylenimine polymer or co-polymer backbone are substituted by an alkyl group which is substituted by at least two different groups selected from OR, COOM, halogen, CONR′R, NR′R, SO₃M, SO₃H, phosphonic acid, phosphonate salt, ammonium salt or a group of the formulae

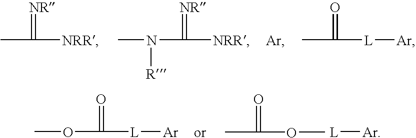

The ethylenimine polymer or co-polymer may be substituted by moieties that provide different activities. For example, the polymer may bear substituents that render the polymer anti-bacterial and other substituents that render the polymer anti-fungal.

In one embodiment of the invention a single ethylenimine polymer, co-polymer or oligomer comprises at least two different substituents wherein each of the substituents provides a different anti-microbial activity, for example, the N atoms of the polymer bear two different substituents, each substituent conferring a different activity.

In another embodiment, a single N atom substituent bears at least two different groups conferring different activities, for example, N atoms are substituted by an alkyl group which alkyl group is substituted by two moieties, one moiety conferring anti-bacterial activity and another moiety conferring anti-fungal activity.

In another embodiment, at least two different inventive ethylenimine polymers, co-polymer or oligomer are blended.

In another embodiment, an inventive ethylenimine polymer, co-polymer or oligomer is blended with another anti-microbial compound.

In addition to the above described inventive substituents, substituents not described above, for example, simple alkyl substituents such as C₁₋₂₄ alkyl or C₁₋₂₄ alkyl substituted by hydroxy, carboxy or carboxylic ester groups may also be present as additional substituents on the N atoms of the ethylenimine polymer or co-polymer.

For example, the invention also pertains to an antimicrobial ethylenimine polymer or co-polymer according to claim 1, wherein 10-99% of the N atoms of the ethylenimine polymer or co-polymer backbone are substituted as described above and at least 1% of the N atoms of the ethylenimine polymer or co-polymer backbone are substituted by C₁₋₂₄ alkyl or said alkyl substituted by —OR, COOR, COOM wherein R and M are as described above.

Any of the N atoms of the ethylenimine polymer or co-polymer backbone may also be substituted more than once by the same substituent, or substituted by more than one substituent.

For example, a portion of the N atoms of the polymer or co-polymer backbone can be substituted by benzyl, a portion by chlorobenzyl and a portion substituted by both benzyl and chlorobenzyl. For example, 10% or more of the N atoms of the polymer or co-polymer backbone can be substituted by an alkyl chain substituted by a hydroxy group and an ammonium cation and a portion of the remaining backbone N atoms by a simple alkyl.

The substituted ethylenimine polymers or co-polymers of the present invention are readily prepared by substituting the N atoms of a pre-existing polymer via known reactions as discussed below, or by substituting the N atoms of ethylenimine monomer or oligomers prior to performing a subsequent polymerization. Such pre-existing polymers are commercially available as are “prepolymers”, that is ethylenimine monomer or oligomers that can be polymerized.

The polymer or co-polymer prior to substitution has a molecular weight in the range of 300 to 50000, typically 400 to 50,000, for example 400 to 5,000, and can be branched or unbranched. The polymer may be also be inherently crosslinked, i.e., crosslinked through reactions of ethylenimine based materials, or cross-linked by agents such as epichlorohydrin, diepoxides, epoxy resins or anhydrides.

The N atoms of an existing ethylenimine polymer, co-polymer or oligomer to be substituted according to the invention may be mono-, di-, or tri-substituted amines depending on the amount of branching and crosslinking. Each N atom may therefore bear one or more of the inventive or non-inventive substituents and the polymer or co-polymer may be cationic.

The N atoms of an ethylenimine polymer, co-polymer or oligomer are substituted by any of the well known substitution reactions of amines. For example, amines can be alkylated, arylated or substituted by heterocycles via reaction with alkyl, aryl or heterocyclic halides, sulfonates, epoxides, etc. under the appropriate conditions, typically in the presence of a base. Alkylation of amines also occurs via addition across a double bond as in reactions with vinyl esters, amides, nitriles sulphones etc. Amines can be acylated by reaction with acid halides, esters, anhydrides, carboxylic acids etc. A variety of metal catalyzed reactions, such as Heck and Suzuki reactions, are also known to derivatise amines.

The reaction conditions will determine the amount of N atoms of the polymer or co-polymer backbone substituted. For example, when alkylating the N atom with an alkyl halide, the amount of alkyl halide used in the reaction represents an upper limit of the amount of alkylating reagent that can be incorporated. As stated above, the N atoms of the polymer or co-polymer backbone may be substituted more than once under the reaction conditions. Therefore, the amount of substituting reagent used in such a reaction will typically be chosen to provide a substitution ratio the range of 0.2 to 2 molar equivalent of substituent per polymer containing N atom. The alkyl halides are items of commerce or readily prepared via known means.

For example, depending on conditions, reaction of a polyethylenimine with benzyl bromide will generate a substituted polymer containing the following moieties 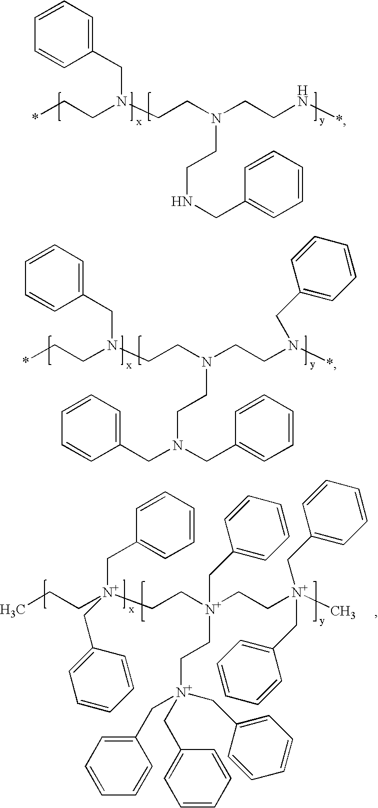
and other benzyl amino moieties included secondary and primary benzyl amino.

The antimicrobial ethylenimine polymers or co-polymers of the invention exhibit pro-nounced antimicrobial action, for example, against pathogenic gram-positive and gram-negative bacteria and against bacteria of the skin flora, and also against yeasts and molds. They are accordingly suitable for disinfection, deodorisation, and for general and antimicrobial treatment of the skin and mucosa and of integumentary appendages (hair), for example, for the disinfection of hands and wounds.

They are accordingly suitable as antimicrobial active substances and preservatives in personal care preparations, for example shampoos, bath additives, hair care preparations, liquid and solid soaps (based on synthetic surfactants and salts of saturated and/or unsaturated fatty acids), lotions and creams, deodorants, other aqueous or alcoholic solutions, e.g. cleansing solutions for the skin, moist cleaning cloths, oils or powders.

The invention accordingly relates also to a personal care preparation comprising at least one of the inventive antimicrobial ethylenimine polymer or co-polymer and cosmetically tolerable carriers or adjuvants.

The personal care preparation according to the invention contains from 0.01 to 15% by weight, for example, from 0.1 to 10% by weight, based on the total weight of the inventive composition, of an inventive antimicrobial ethylenimine polymer or co-polymer, and cosmetically tolerable adjuvants.

Depending upon the form of the personal care preparation, it comprises, in addition to the antimicrobial ethylenimine polymer or co-polymer further constituents, for example sequestering agents, colourings, perfume oils, thickening or solidifying agents (consistency regulators), emollients, UV-absorbers, skin protective agents, antioxidants, additives that improve the mechanical properties, such as dicarboxylic acids and/or aluminium, zinc, calcium or magnesium salts of C₁₄-C₂₂ fatty acids, and, optionally, preservatives.

The personal care preparation according to the invention may be in the form of a water-in-oil or oil-in-water emulsion, an alcoholic or alcohol-containing formulation, a vesicular dispersion of an ionic or non-ionic amphiphilic lipid, a gel, a solid stick or an aerosol formulation.

As a water-in-oil or oil-in-water emulsion, the cosmetically tolerable adjuvant contains preferably from 5 to 50% of an oil phase, from 5 to 20% of an emulsifier and from 30 to 90% water. The oil phase may comprise any oil suitable for cosmetic formulations, for example one or more hydrocarbon oils, a wax, a natural oil, a silicone oil, a fatty acid ester or a fatty alcohol. Preferred mono- or poly-ols are ethanol, isopropanol, propylene glycol, hexylene glycol, glycerol and sorbitol.

Cosmetic formulations according to the invention are used in various fields. There come into consideration, for example, the following preparations:
skin-care preparations, e.g. skin-washing and cleansing preparations in the form of tablet-form or liquid soaps, synthetic detergents or washing pastes,
bath preparations, e.g. liquid (foam baths, milks, shower preparations) or solid bath preparations, e.g. bath cubes and bath salts;
skin-care preparations, e.g. skin emulsions, multi-emulsions or skin oils;
cosmetic personal care preparations, e.g. facial make-up in the form of day creams or powder creams, face powder (loose or pressed), rouge or cream make-up, eye-care preparations, e.g. eyeshadow preparations, mascaras, eyeliners, eye creams or eye-fix creams; lip-care preparations, e.g. lipsticks, lip gloss, lip contour pencils, nail-care preparations, such as nail varnish, nail varnish removers, nail hardeners or cuticle removers;
intimate hygiene preparations, e.g. intimate washing lotions or intimate sprays;
foot-care preparations, e.g. foot baths, foot powders, foot creams or foot balsams, special deodorants and antiperspirants or callus-removing preparations;
light-protective preparations, such as sun milks, lotions, creams or oils, sun-blocks or tropicals, pre-tanning preparations or after-sun preparations;
skin-tanning preparations, e.g. self-tanning creams;
depigmenting preparations, e.g. preparations for bleaching the skin or skin-lightening preparations;
insect-repellents, e.g. insect-repellent oils, lotions, sprays or sticks;
deodorants, such as deodorant sprays, pump-action sprays, deodorant gels, sticks or roll-ons;
antiperspirants, e.g. antiperspirant sticks, creams or roll-ons;
preparations for cleansing and caring for blemished skin, e.g. synthetic detergents (solid or liquid), peeling or scrub preparations or peeling masks;
hair-removal preparations in chemical form (depilation), e.g. hair-removing powders, liquid hair-removing preparations, cream- or paste-form hair-removing preparations, hair-removing preparations in gel form or aerosol foams;
shaving preparations, e.g. shaving soap, foaming shaving creams, non-foaming shaving creams, foams and gels, preshave preparations for dry shaving, aftershaves or aftershave lotions;
fragrance preparations, e.g. fragrances (eau de Cologne, eau de toilette, eau de parfum, parfum de toilette, perfume), perfume oils or perfume creams;
dental care, denture-care and mouth-care preparations, e.g. toothpastes, gel toothpastes, tooth powders, mouthwash concentrates, anti-plaque mouthwashes, denture cleaners or denture fixatives;
cosmetic hair-treatment preparations, e.g. hair-washing preparations in the form of shampoos and conditioners, hair-care preparations, e.g. pretreatment preparations, hair tonics, styling creams, styling gels, pomades, hair rinses, treatment packs, intensive hair treatments, hair-structuring preparations, e.g. hair-waving preparations for permanent waves (hot wave, mild wave, cold wave), hair-straightening preparations, liquid hair-setting preparations, hair foams, hairsprays, bleaching preparations, e.g. hydrogen peroxide solutions, lightening shampoos, bleaching creams, bleaching powders, bleaching pastes or oils, temporary, semi-permanent or permanent hair colorants, preparations containing self-oxidising dyes, or natural hair colorants, such as henna or camomile.

An antimicrobial soap has, for example, the following composition:
0.01 to 5% by weight of the instant antimicrobial polymer,
0.3 to 1% by weight titanium dioxide,
1 to 10% by weight stearic acid,
soap base ad 100%, e.g. a sodium salt of tallow fatty acid or coconut fatty acid, or glycerol.

A shampoo has, for example, the following composition:
0.01 to 5% by weight of the instant antimicrobial polymer,
12.0% by weight sodium laureth-2-sulfate,
4.0% by weight cocamidopropyl betaine,
3.0% by weight NaCl and
water ad 100%.

A deodorant has, for example, the following composition:
0.01 to 5% by weight of the instant antimicrobial polymer,
60% by weight ethanol,
0.3% by weight perfume oil, and
water ad 100%.

The invention relates also to an oral composition containing from 0.01 to 15% by weight, based on the total weight of the composition, of the instant antimicrobial polymer, and orally tolerable adjuvants.

Example of an oral composition:
10% by weight sorbitol,
10% by weight glycerol,
15% by weight ethanol,
15% by weight propylene glycol,
0.5% by weight sodium lauryl sulfate,
0.25% by weight sodium methylcocyl taurate,
0.25% by weight polyoxypropylene/polyoxyethylene block copolymer,
0.10% by weight peppermint flavouring,
0.1 to 0.5% by weight of a compound of formula (1), and
48.6% by weight water.

The oral composition according to the invention may be, for example, in the form of a gel, a paste, a cream or an aqueous preparation (mouthwash).

The oral composition according to the invention may also comprise compounds that release fluoride ions which are effective against the formation of caries, for example inorganic fluoride salts, e.g. sodium, potassium, ammonium or calcium fluoride, or organic fluoride salts, e.g. amine fluorides, which are known under the trade name Olafluor.

The antimicrobial ethylenimine polymers or co-polymers of this invention are also suitable for treating, especially preserving, textile fibre materials. Such materials are undyed and dyed or printed fibre materials, e.g. of silk, wool, polyamide or polyurethanes, and especially cellulosic fibre materials of all kinds. Such fibre materials are, for example, natural cellulose fibres, such as cotton, linen, jute and hemp, as well as cellulose and regenerated cellulose.

The antimicrobial ethylenimine polymers or co-polymers of this invention are suitable also for treating, especially imparting antimicrobial properties to or preserving, plastics, e.g. polyethylene, polypropylene, polyurethane, polyester, polyamide, polycarbonate, latex etc. Fields of use therefore are, for example, floor coverings, plastics coatings, plastics containers and packaging materials; kitchen and bathroom utensils (e.g. brushes, shower curtains, sponges, bathmats), latex, filter materials (air and water filters), plastics articles used in the field of medicine, e.g. dressing materials, syringes, catheters etc., so-called “medical devices”, gloves and mattresses.

The antimicrobial ethylenimine polymers or co-polymers of this invention are suitable also for treating, especially imparting antimicrobial properties to or preserving industrial formulations such as coatings, lubricants etc.

Paper, for example papers used for hygiene purposes, may also be provided with antimicrobial properties using the antimicrobial ethylenimine polymers or co-polymers of this invention.

It is also possible for nonwovens, e.g. nappies/diapers, sanitary towels, panty liners, and cloths for hygiene and household uses, to be provided with antimicrobial properties in accordance with the invention.

The antimicrobial ethylenimine polymers or co-polymers of this invention are also used in washing and cleaning formulations, e.g. in liquid or powder washing agents or softeners.

The antimicrobial ethylenimine polymers or co-polymers of this invention can also be used especially in household and general-purpose cleaners for cleaning and disinfecting hard surfaces.

A cleaning preparation has, for example the following composition:
0.01 to 5% by weight of a compound of formula (1)
3.0% by weight octyl alcohol 4EO
1.3% by weight fatty alcohol C₈-C₁₀polyglucoside
3.0% by weight isopropanol
water ad 100%.

In addition to preserving cosmetic and household products, the preservation of technical products, the provision of technical products with antimicrobial properties and use as a biocide in technical processes are also possible, for example in paper treatment, especially in paper treatment liquors, printing thickeners of starch or cellulose derivatives, surface-coatings and paints.

The antimicrobial ethylenimine polymers or co-polymers of the invention are also suitable for the antimicrobial treatment of wood and for the antimicrobial treatment of leather, the preserving of leather and the provision of leather with antimicrobial properties.

The compounds according to the invention are also suitable for the protection of cosmetic products and household products from microbial damage.

Co-pending application 60/720,662, which is hereby incorporated in its entirety by reference, discloses compounds useful in coatings or films in protecting surfaces from bio-fouling. Such surfaces include surfaces in contact with marine environments (including fresh water, brackish water and salt water environments), for example, the hulls of ships, surfaces of docks or the inside of pipes in circulating or pass-through water systems. Other surfaces are susceptible to similar biofouling, for example walls exposed to rain water, walls of showers, roofs, gutters, pool areas, saunas, floors and walls exposed to damp environs such as basements or garages and even the housing of tools and outdoor furniture.

The antimicrobial ethylenimine polymers or co-polymers of this invention are also useful in preventing bio-fouling, or eliminating or controlling microbe accumulation on the surfaces described in co-pending application 60/720,662 either by incorporating the antimicrobial ethylenimine polymers or co-polymers into the article or surface of the article in question or by applying the antimicrobial ethylenimine polymers or co-polymers to these surfaces either directly or as part of a coating or film as described in co-pending application 60/720,662.

When applied as a part of a film or coating, the antimicrobial ethylenimine polymers or co-polymers of this invention are part of a composition which also comprises a binder.

The binder may be any polymer or oligomer compatible with the present antimicrobials. The binder may be in the form of a polymer or oligomer prior to preparation of the anti-fouling composition, or may form by polymerization during or after preparation, including after application to the substrate. In certain applications, such as certain coating applications, it will be desirable to crosslink the oligomer or polymer of the anti fouling composition after application.

The term binder as used in the present invention also includes materials such as glycols, oils, waxes and surfactants commercially used in the care of wood, plastic, glass and other surfaces. Examples include water proofing materials for wood, vinyl protectants, protective waxes and the like.

The composition may be a coating or a film. When the composition is a thermoplastic film which is applied to a surface, for example, by the use of an adhesive or by melt applications including calendaring and co-extrusion, the binder is the thermoplastic polymer matrix used to prepare the film.

When the composition is a coating, it may be applied as a liquid solution or suspension, a paste, gel, oil or the coating composition may be a solid, for example a powder coating which is subsequently cured by heat, UV light or other method.

As the composition of the invention may be a coating or a film, the binder can be comprised of any polymer used in coating formulations or film preparation. For example, the binder is a thermoset, thermoplastic, elastomeric, inherently crosslinked or crosslinked polymer.

Thermoset, thermoplastic, elastomeric, inherently crosslinked or crosslinked polymers include polyolefin, polyamide, polyurethane, polyacrylate, polyacrylamide, polycarbonate, polystyrene, polyvinyl acetates, polyvinyl alcohols, polyester, halogenated vinyl polymers such as PVC, natural and synthetic rubbers, alkyd resins, epoxy resins, unsaturated polyesters, unsaturated polyamides, polyimides, silicon containing and carbamate polymers, fluorinated polymers, crosslinkable acrylic resins derived from substituted acrylic esters, e.g. from epoxy acrylates, urethane acrylates or polyester acrylates. The polymers may also be blends and copolymers of the preceding chemistries.

Biocompatible coating polymers, such as, poly[-alkoxyalkanoate-co-3-hydroxyalkenoate] (PHAE) polyesters, Geiger et. al. Polymer Bulletin 52, 65-70 (2004), can also serve as binders in the present invention.

Alkyd resins, polyesters, polyurethanes, epoxy resins, silicone containing polymers, fluorinated polymers and polymers of vinyl acetate, vinyl alcohol and vinyl amine are non-limiting examples of common coating binders useful in the present invention. Other coating binders, of course, are part of the present invention.

Coatings are frequently crosslinked with, for example, melamine resins, urea resins, isocyanates, isocyanurates, polyisocyanates, epoxy resins, anhydrides, poly acids and amines, with or without accelerators.

The compositions of present invention are for example a coating applied to a surface which is exposed to conditions favorable for bioaccumulation. The presence of the antimicrobial ethylenimine polymers or co-polymers of this invention in said coating will prevent the adherence of organisms to the surface.

The anti-microbial compound of the present invention may be part of a complete coating or paint formulation, such as a marine gel-coat, shellac, varnish, lacquer or paint, or the anti fouling composition may comprise only a polymer of the instant invention and binder, or a polymer of the instant invention, binder and a carrier substance. It is anticipated that other additives encountered in such coating formulations or applications will find optional use in the present applications as well.

The coating may be solvent borne or aqueous. Aqueous are typically considered more environmentally friendly.

The coating is, for example, aqueous dispersion of a polymer of the instant invention and a binder or a water based coating or paint. For example, the coating comprises an aqueous dispersion of a polymer of the instant invention and an acrylic, methacrylic or acrylamide polymers or co-polymers or a poly[-alkoxyalkanoate-co-3-hydroxyalkenoate] polyester.

The coating is, for example, a coating or varnish used in marine applications.

The coating may be applied to a surface which has already been coated, such as a protective coating, a clear coat or a protective wax applied over a previously coated article.

Coating systems include marine coatings, wood coatings, other coatings for metals and coatings over plastics and ceramics. Exemplary of marine coatings are gel coats comprising an unsaturated polyester, a styrene and a catalyst.

The coating is, for example a house paint, or other decorative or protective paint. It may be a paint or other coating that is applied to cement, concrete or other masonry article. The coating may be a water proofer as for a basement or foundation.

As the anti-fouling composition is intended for use in maritime applications as well as near pool areas etc., the composition may be part of a non-skid coating including coatings for stairs, paths and handrails.

The coating composition is applied to a surface by any conventional means including spin coating, dip coating, spray coating, draw down, or by brush, roller or other applicator. A drying or curing period will typically be needed.

Coating or film thickness will vary depending on application and will become apparent to one skilled in the art after limited testing.

The composition may be in the form of a protective laminate film.

Such a film typically comprises thermoset, thermoplastic, elastomeric, or crosslinked polymers. Examples of such polymers include, but are not limited to, polyolefin, polyamide, polyurethane, polyacrylate, polyacrylamide, polycarbonate, polystyrene, polyvinyl acetates, polyvinyl alcohols, polyester, halogenated vinyl polymers such as PVC, natural and synthetic rubbers, alkyd resins, epoxy resins, unsaturated polyesters, unsaturated polyamides, polyimides, fluorinated polymers, silicon containing and carbamate polymers. The polymers may also be blends and copolymers of the preceding chemistries.

When the anti-fouling composition is a preformed film it is applied to the surface by, for example, the use of an adhesive, or co-extruded onto the surface. It may also be mechanically affixed via fasteners which may require the use of a sealant or caulk wherein the esters of the instant invention may also be advantageously employed.

A plastic film may also be applied with heat which includes calendaring, melt applications and shrink wrapping.

The composition may be part of a polish, such a furniture polish, or a dispersant or surfactant formulation such as a glycol or mineral oil dispersion or other formulation as used in for example wood protection.

Examples of useful surfactants include, but are not limited to, polyoxyethylene-based surface-active substances, including polyoxyethylene sorbitan tetraoleate (PST), polyoxyethylene sorbitol hexaoleate (PSH), polyoxyethylene 6 tridecyl ether, polyoxyethylene 12 tridecyl ether, polyoxyethylene 18 tridecyl ether, TWEEN® surfactants, TRITON® surfactants, and the polyoxyethlene-polyoxypropylene copolymers such as the PLURONIC® and POLOXAMER® product series (from BASF). Other matrix-forming components include dextrans, linear PEG molecules (MW 500 to 5,000,000), star-shaped PEG molecules, comb-shaped and dendrimeric, hyperbrached PEG molecules, as well as the analogous linear, star, and dendrimer polyamine polymers, and various carbonated, perfluorinated (e.g., DUPONT ZONYL® fluorosurfactants) and siliconated (e.g, dimethylsiloxane-ethylene oxide block copolymers) surfactants.

Given the wide array of applications for the present anti-microbial compositions, the composition may contain other additives such as antioxidants, UV absorbers, hindered amines, phosphites or phosphonites, benzofuran-2-ones, thiosynergists, polyamide stabilizers, metal stearates, nucleating agents, fillers, reinforcing agents, lubricants, emulsifiers, dyes, pigments, dispersants, other optical brighteners, flame retardants, antistatic agents, blowing agents and the like, such as the materials listed below, or mixtures thereof.

The substrate can be an inorganic or organic substrate, for example, a metal or metal alloy; a thermoplastic, elastomeric, inherently crosslinked or crosslinked polymer as described above; a natural polymer such as wood or rubber; a ceramic material; glass; leather or other textile.

The substrate may be, for example, non-metal inorganic surfaces such as silica, silicon dioxide, titanium oxides, aluminum oxides, iron oxides, carbon, silicon, various silicates and sol-gels, masonry, and composite materials such as fiberglass and plastic lumber (a blend of polymers and wood shavings, wood flour or other wood particles).

The inorganic or organic substrate is, for example, a metal or metal alloy, a thermoplastic, elastomeric, inherently crosslinked or crosslinked polymer, a ceramic material or a glass.

The substrate may be a multi-layered article comprised of the same or different components in each layer. The surface coated or laminated may be the exposed surface of an already applied coating or laminate.

The inorganic or organic substrate to be coated or laminated can be in any solid form.

For example, polymer substrates may be plastics in the form of films, injection-molded articles, extruded workpieces, fibres, felts or woven fabrics.

For example molded or extruded polymeric articles used in construction or the manufacture of durable goods such as siding, fascia and mailboxes can all benefit from the present method for stabilizer replenishment.

Plastics which would benefit from the present method include, but are not limited to, plastics used in construction or the manufacture of durable goods or machine parts, including outdoor furniture, boats, siding, roofing, glazing, protective films, decals, sealants, composites like plastic lumber and fiber reinforced composites, functional films including films used in displays as well as articles constructed from synthetic fibers such as awnings, fabrics such as used in canvas or sails and rubber articles such as outdoor matting and other uses cited in this disclosure. Exemplary of such plastics are polypropylene, polyethylene, PVC, POM, polysulfones, styrenics, polyamides, urethanes, polyesters, polycarbonate, acrylics, butadiene, thermoplastic polyolefins, ionomers, unsaturated polyesters and blends of polymer resins including ABS, SAN and PC/ABS.

Examples of applications of the compositions of the instant invention are surface coatings, protective paints, other coatings and laminates applied to vulnerable surfaces, for example, the hulls of ships, surfaces of docks or the inside of pipes in circulating or pass-through water systems, walls exposed to rain water, walls of showers, roofs, gutters, pool areas, saunas, floors and walls exposed to damp environs such as basements or garages, the housing of tools and outdoor furniture.

For example, the compositions of the instant invention are found, among other places, on the surfaces of:

boat hulls, docks, buoys, drilling platforms, ballast water tanks,

machines, machine parts, recreational, air conditioning systems, ion exchangers, process water systems, other industrial water systems, solar-powered units, heat exchangers, sump pumps, drainage systems,

roofing, basements, walls, facades, greenhouses, sheds, storage areas, awnings, garden fencing, wood protection, tent roof material, fabrics, outdoor furniture, door mats,

public conveniences, bathrooms, showers, swimming pools, saunas, jointing, sealing compounds, public conveyances, locker rooms etc.

Process water includes any process water stream which is used for heating or cooling purposes in closed or open circulating systems.

Particular embodiments of the invention therefore relate to

methods for protecting plastics, coatings, home or personal care formulations, industrial formulations or technical process against the action of microbes which comprises adding an effective amount of the present polymer or copolymer to the formulation or process;

a method for protecting skin, mucosa and integumentary appendages against the action of microbes which comprises applying a preparation comprising an effective amount of the present polymer or copolymer;

a method for protecting paper, wood, leather, synthetic textile materials or natural textile materials such as cotton against the action of microbes comprising incorporating or applying an effective amount of the present polymer or copolymer or a composition comprising an effective amount the present polymer or copolymer;

a method for cleaning and disinfecting hard surfaces which comprises applying a preparation comprising an effective amount of the present polymer or copolymer;

a method for preventing bio-fouling of an article comprising incorporating the present antimicrobial ethylenimine polymer or co-polymer into the article or surface of the article or by applying the antimicrobial ethylenimine polymer or co-polymer to these surfaces either directly or as part of a coating or film.

The following non-limiting examples illustrate some aspects of the invention.

### WORKING EXAMPLES

### Example 1

To a solution of the 1.7 grams of a polyethylenimine polymer in water at 80° C. is added 6.8 grams of benzyl bromide and 5.6 grams of potassium carbonate in 6 mL methanol and 7 mL water and the mixture is stirred until no benzyl bromide is detected by thin layer chromotography (TLC). The solvent is evaporated, the residue is treated with 40 mL of ethanol at reflux for 15 minutes, the mixture is cooled and filtered, and the resulting solution is concentrated and dried under vacuum. Hexane, 40 mL, is added and the mixture is heated to by reflux giving 2 phases which are separated, the oily layer containing the polymer is separated from the hexane layer and evaporated to dryness under vacuum to give 3.22 grams of benzyl substituted polyethylenimine as a yellow solid. Analysis by NMR reveals 1.3-1.5 equivalents of benzyl groups per ethylenimine monomer group and a 5-7% of benzyl alcohol as by-product.

### Example 2

Polyethylenimine is also benzylated by heating to reflux a mixture of 5 grams of polyethylenimine, 10 grams of benzyl bromide, 3.8 grams of potassium hydroxide and 50 ml of ethanol until no benzyl bromide can be detected by TLC. The reaction mixture was filtered and the solution was concentrated and dried under vacuum to give 7.8 grams of benzyl substituted polyethylenimine as a yellow syrup.

### Example 3

A mixture of 5.6 grams of polyethylenimine and 2.9 grams cyanamide and 100 mL toluene are stirred at reflux for 12 hours. The solvent is removed under vacuum to leave 8.4 grams of a guanidine substituted polyethylenimine as a solid, structure confirmation by ¹³C NMR.

Using a variant of the general procedure of Example 1 or 2, polyethylenimine polymers are reacted with the following halides (RX) to generate the corresponding N-substituted polymer. The halides are items of commerce or readily prepared via known means.

| Ex- | | RX per | Substitution |
|---|---|---|---|
| ample | RX | monomer | per monomer |
| 4 | 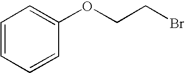 | 0.25 | 0.24 |
| 5 | 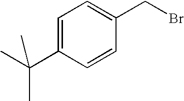 | 0.25 | 0.25 |
| 6 | 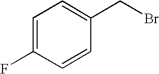 | 0.25 | 0.25 |
| 7 | 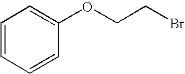 | 0.5 | 0.36 |
| 8 | 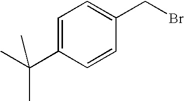 | 0.5 | 0.38 |
| 9 | 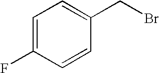 | 0.5 | 0.45 |
| 10 | 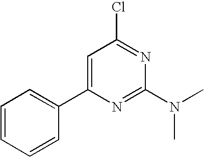 | 0.5 | 0.25 eq |
| 11 | 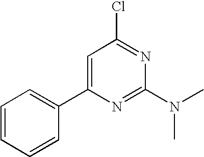 | 1 | 0.23 eq |
| 12 | 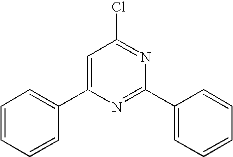 | 0.5 | 0.49 eq |
| 13 | 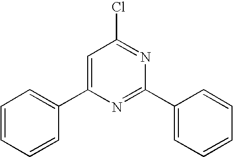 | 1 | 0.60 eq |
| 14 | 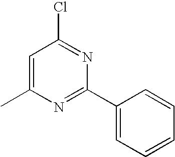 | 0.25 | 0.23 eq |
| 15 | 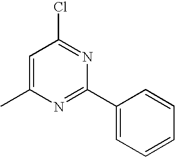 | 0.5 | 0.44 eq |

RX is the halide used
RX per monomer is the ratio of halide used for each ethylenimine monomer unit (—CH2-CH2-NR—) of the starting polymer
Substitution per monomer is the percentage of polymer backbone N atoms substituted in the resulting product

Using a variant of the general procedure of Example 1 or 2, three different polyethylenimine polymers with molecular weights of 800, 2,000 and/or 25,000 are substituted with the following substituents using from 0.25 to 2 equivalents of the appropriate halides (RX) per monomer to generate the corresponding N-substituted polymer and the resulting polymers are tested for activity against bacteria, e. coloi, s. aureus; fungi, a. pull, p. funic, a. niger, adhesion of microbes or biofilm accumulation. All compounds are effective in at least one test; some are effective in more than one or all of the tests.

| Example | Substituent |
|---|---|
| 16 | 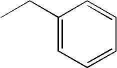 |
| 17 | 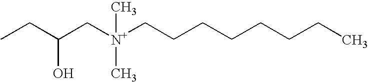 |
| 18 | 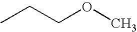 |
| 19 | 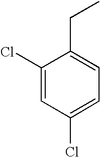 |
| 20 | 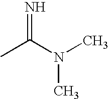 |
| 21 | 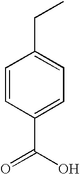 |
| 22 | 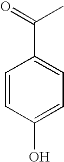 |
| 23 | 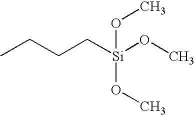 |
| 24 | 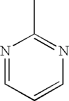 |
| 25 | 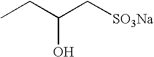 |
| 26 | 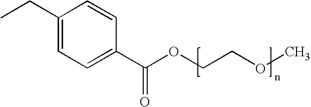 |
| 27 | 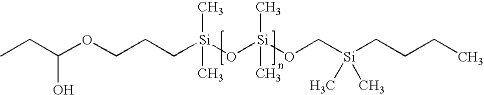 |
| 28 | 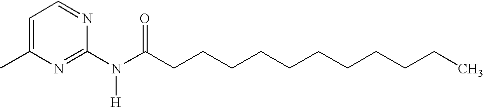 |

Using a variant of the general procedure of Example 1 or 2, polyethylenimine polymers are substituted with the following pairs of substituents, using 0.5 or 1 equivalent of the appropriate halides (RX) per monomer, the substituent halides are added in a 1:1 ratio relative to each other in each example, to generate the corresponding N-substituted polymer and the resulting polymers are tested for activity against bacteria, e. coloi, s. aureus; fungi, a. pull, p. funic, a. niger, adhesion of microbes or biofilm accumulation. All compounds are effective in at least one test; some are effective in more than one or all of the tests.

| Example | Substituent 1 | Substituent 2 |
|---|---|---|
| 29 | 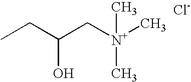 | Benzyl |
| 30 | 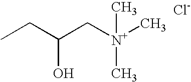 | Hexyl |
| 31 | 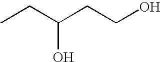 | Benzyl |
| 32 | Benzyl | Dodecyl |
| 33 | 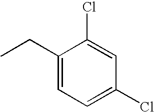 | 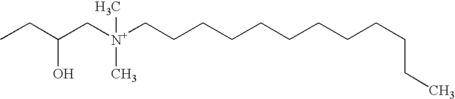 |
| 34 | 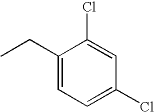 | Dodecyl |
| 35 | Dodecyl | 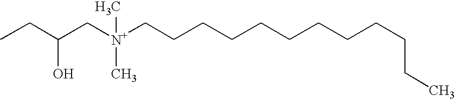 |
| 36 | Benzyl | 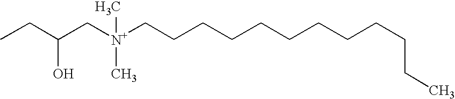 |
| 37 | Benzyl | 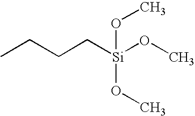 |
| 38 | Dodecyl | 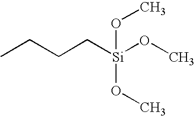 |

Microbiological activity of the PEIs:
Microbicidal Activity:

Microbicidal activity is tested according to trivial modifications of the standard EN1040 test method. A bacterial suspension with a cell count of about 10⁷ cfu/ml is contacted with appropriate concentrations of the specific substances and the residual cell count is determined after incubation times of 5 and 30 min. at room temperature under continuous stirring. Staphylococcus aureus is tested as gram+ and Escherichia coli as gram-organism. The resulting cell count reduction is compared to a water control.

Fungicidal Activity:

Fungicidal activity is tested according to trivial modifications of the standard EN12175 test method. A fungal spore suspension with a spore cell count of about 10⁶ cfu/ml is contacted with appropriate concentrations of the specific substances and the residual spore cell count is determined after incubation times of 30 and 60 min. at room temperature under continuous stirring. Penicillium funiculosum, Aspergillus niger and Aureobasidium pullulans are tested as important mold strains. The resulting cell count reduction is compared to a water control.

Polyethylenimine samples, for example functionalized by Quab 342, Quab 426, i.e., 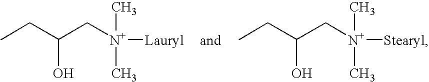
benzyl bromide, hexyl bromide and dodecyl bromide, all show full microbiocidal and fungicidal activity.
Biofilm Inhibition:

The ability of the compounds for inhibiting the initial stages of biofilm formation is tested in a microplate based screening assay. Standard test specimen of polycarbonate are contacted with compound solutions in water or ethanol at a concentration of 0.5% for ½ hour for the compounds to form a film on the pin surface. The pins are then dried at room temperature under laminar flow. The coated pins are contacted with a bacterial inoculum of Staphylococcus aureus at a cell count of 10⁴-10⁵ cfu/ml in a microplate and a biofilm is allowed to form on the plastic surface over 24 hours. Loosely attached cells are then rinsed off in a couple of rinsing steps, then the biofilm on the surface is removed by ultrasonic treatment. The eluted cells are transferred into new microplates in Caso broth and growth is followed by measurement of optical density at 620 nm over 24 hours. The results are evaluated as growth curves of the eluted cells over 24 hours incubation time in comparison to the growth curve of untreated samples.

Polyethylenimine samples, for example functionalized by Quab 342, Quab 426, benzyl bromide and hexyl bromide, show full biofilm inhibition in the screening assay described above.

We claim:

1. An antimicrobial ethylenimine polymer or co-polymer, wherein 10-100% of the nitrogen atoms of the polymer or co-polymer backbone are substituted by one or more substituents a-d:
a) C₁₋₂₄ alkyl, C₃₋₂₄ alkenyl, C₁₋₂₄ alkylcarbonyl or C₃₋₂₄ alkenylcarbonyl which are uninterrupted or interrupted one or more times by one or more oxygen atoms, sulfur atoms, —SO— or —SO₂—, and which are substituted one or more times by one or more moieties C₃₋₆ cycloalkyl, —OR, —COOR, —COOM, —SO₃M, —SO₃H, phosphonic acid, halogen, —CONR′R, —NR′R, phosphonate salt, ammonium salt or group of the formulae 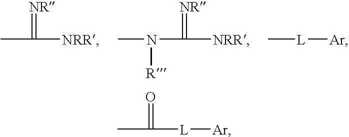
or a group —Si(G)₃ wherein each G is independently hydroxyl, C₁₋₄ alkyl or C₁₋₄ alkoxy,
with the proviso that uninterrupted C₁₋₂₄ alkyl is not substituted by biguamide, C₃₋₆ cycloalkyl, —COOM, —COOR where R is an unsubstituted alkyl, —OR where R is H or unsubstituted alkylcarbonyl or —CONR′R unless at least one other of the substituents is also present;
b) heterocycle of the formulae 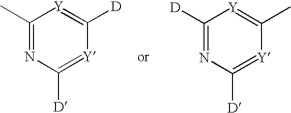
wherein Y and Y′ are independently N, C—R, C—OR or C—NRR′ and D and D′ are independently R, —OR or —NRR′;
c) group of the formulae 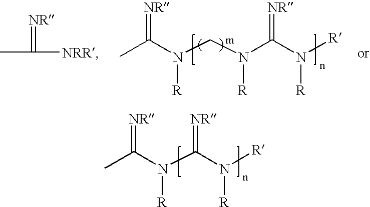
wherein m and n independently are 1, 2, 3, 4, 5 or 6;
or
d)-L-Poly where Poly is branched or unbranched polymer or oligomer selected from polyether, polysiloxane, styrenic polymer or polyol;
wherein
R, R′ and R″, independently of each other are hydrogen;
a group -L-Ar, 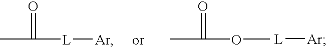
C₁₋₂₄ alkyl, C₃₋₂₄ alkenyl, C₃₋₆ cycloalkyl or C₁₋₂₄ alkylcarbonyl which are uninterrupted or interrupted one or more times by one or more oxygen atoms, sulfur atoms, carbonyl, —COO—, —CONH—, —NH—, —CON(C₁₋₈ alkyl)- or —N(C₁₋₈ alkyl)-, which uninterrupted or interrupted alkyl, alkenyl, cycloalkyl or alkylcarbonyl are unsubstituted or substituted one or more times by one or more halogen, —OH, C₇₋₁₂ aralkyl, C₂₋₁₂alkylcarbonyl, C₁₋₂₄alkoxy, C₂₋₂₄alkylcarboxy, —COOM, —CONH₂, —CON(H)(C₁₋₈alkyl), —CON(C₁₋₈alkyl)₂, —NH₂, —N(H)(C₁₋₈ alkyl), —N(C₁₋₁₈ alkyl)₂, —SO₃M, phenyl, phenyl substituted one or more times by one or more C₁₋₈ alkyl, naphthyl, naphthyl substituted one or more times by one or more C₁₋₈alkyl, purine, pyridine, pyrimidine, triazine or imidazole which purine, pyridine, pyrimidine, triazine or imidazole are unsubstituted or substituted by one or more C₁₋₁₂ alkyl wherein the purine, pyridine, pyrimidine, triazine or imidazole is neutral or ionically charged, amidine, guanidine, ammonium salt, phosphonic acid, phosphonate salt or a group 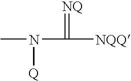
wherein each Q or Q′ is independently hydrogen, C₁₋₁₂alkyl, phenyl or benzyl;
or
when attached to a nitrogen atom, R and R′, together with the nitrogen atom to which they are attached, form a 5-, 6- or 7-membered ring which is uninterrupted or interrupted by —O—, —NH— or —N(C₁₋₁₂ alkyl)-;
L is a direct bond, C₁₋₂ alkylene which is uninterrupted or interrupted by one or more oxygen atoms and which is unsubstituted or substituted one or more times by one or more —OH, C₁₋₈ alkyl, C₁₋₂₄ alkoxy, C₂₋₂₄alkylcarboxy, —NH₂, —N(H)(C₁₋₈alkyl), —N(C₁₋₈ alkyl)₂ or ammonium salt:
Ar is C₆₋₁₀ aromatic or C₁₋₉ saturated or unsaturated heterocycle which are unsubstituted or substituted one or more times by one or more halogen, —OH, C₁₋₂₄ alkoxy, C₂₋₂₄ alkylcarboxy, —COOQ″, —CONH₂, —CON(H)(C₁₋₈ alkyl), —CON(C₁₋₈ alkyl)₂, —NH₂, —N(H)(C₁₋₈ alkyl), —N(C₁₋₈ alkyl)₂, —SO₃M, SO₃H, ammonium salt, phosphonic acid, phosphonate salt, C₁₋₂₄ alkyl which is unsubstituted or substituted one or more times by one or more halogen, phenyl which is unsubstituted or substituted by one or more times by one or more C₁₋₈alkyl, naphthyl, purine, pyridine, pyrimidine, triazine or imidazole which purine, pyridine, pyrimidine, triazine or imidazole are unsubstituted or substituted by one or more C₁₋₁₂ alkyl wherein the purine, pyridine, pyrimidine, triazine or imidazole is neutral or is ionically charged; wherein Q″ is hydrogen, metal cation, glycol ether, polysiloxane, phenyl or benzyl, or phenyl or benzyl substituted one or more times by one or more halogen, hydroxy, C₁₋₂₄ alkoxy or C₁₋₁₂ alkyl,
M is a metal cation or an ammonium cation
and when the N atom of the ethylenimine polymer is tetra substituted, it is a cation with a corresponding counter anion.

2. A polymer or co-polymer according to claim 1, wherein at least a portion of the substituents are
C₁₋₂₄ alkyl, C₃₋₂₄ alkenyl, C₁₋₂₄ alkylcarbonyl or C₃₋₂₄ alkenylcarbonyl which are uninterrupted or interrupted one or more times by one or more oxygen atoms, sulfur atoms, —SO— or —SO₂—, and which are substituted one or more times by one or more —OR, —COOR, —COOM, —NR′R, —SO₃M, —SO₃H, halogen, —NR′R, ammonium salt or group of the formulae 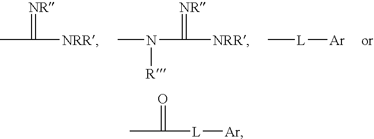
with the proviso that uninterrupted C₁₋₂₄ alkyl is not substituted by biguamide, —COOM, —COOR where R is an unsubstituted alkyl, or —OR where R is H or unsubstituted alkylcarbonyl, unless at least one other of the substituents is also present.

3. A polymer or co-polymer according to claim 2, wherein at least a portion of the substituents are C₁₋₂₄ alkyl or C₁₋₂₄ alkylcarbonyl substituted by one or more halogen, ammonium salt, 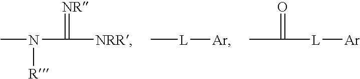
or —OR wherein R is -L-Ar, or

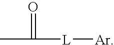

4. A polymer or co-polymer according to claim 2, wherein at least a portion of the substituents are C₁₋₂₄ alkyl substituted one or more times by one or more halogen or ammonium salt, C₁₋₂₄ alkylcarbonyl substituted one or more times by one or more halogen or ammonium salt or at least a portion of the substituents are benzyl, benzoyl or benzyl or benzoyl substituted one or more times by one or more halogens, hydroxyl, C₁₋₁₂ alkyl, C₁₋₁₂ alkoxy or C₁₋₁₂ alkylcarboxy.

5. A polymer or co-polymer according to claim 2, wherein at least a portion of the substituents are C₁₋₂₄ alkyl or C₁₋₂₄ alkylcarbonyl substituted by at least one group selected from ammonium salt, phenoxy, benzyloxy, substituted phenoxy, substituted benzyloxy, benzyl, substituted benzyl, 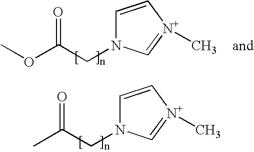
where n is a number from 1 to 12.

6. A polymer or co-polymer according to claim 1, wherein at least a portion of the substituents are one or more groups 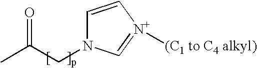
wherein p is 1, 2, 3, 4, 5, or 6.

7. A polymer or co-polymer according to claim 1, wherein at least a portion of the substituents are selected from the group consisting of C₁₋₂₄ alkyl, or C₁₋₂₄ alkylcarbonyl substituted by at least two different moieties.

8. A polymer or co-polymer according to claim 1, wherein at least a portion of the substituents are 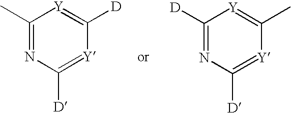
wherein Y and Y′ are independently N, C—R, C—OR or C—NRR′ and
D and D′ are independently R, OR or NRR′.

9. A polymer or co-polymer according to claim 1, wherein at least a portion of the substituents are 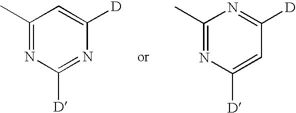
wherein
D and D′ are independently R, OR or NRR′ wherein R and R′ are independently hydrogen, ammonium salt, C₁₋₂₄ alkyl, C₁₋₂₄ alkanoyl which are unsubstituted or substituted one or more times by one or more halogen, hydroxyl or ammonium salt; or R and R′ are independently -L-Ar, 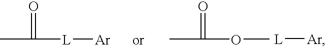
wherein L is a direct bond or C₁₋₁₂ alkylene and
Ar is phenyl or phenyl substituted one or more times by one or more halogen, —OH, C₁₋₂₄ alkoxy, C₂₋₂₄alkylcarboxy, —COOH, —COOM, —CONH₂, —CON(H)(C₁₋₁₂ alkyl), —CON(C₁₋₁₂ alkyl)₂, —NH₂, —N(H)(C₁₋₂ alkyl), —N(C₁₋₁₂ alkyl)₂, ammonium salt, C₁₋₁₂ alkyl or alkyl substituted one or more times by one or more halogen.

10. A polymer or co-polymer according to claim 1, wherein at least a portion of the substituents are selected from the group consisting of -L-Poly where Poly is branched or unbranched polymer or oligomer selected from polyether and polysiloxane.

11. An antimicrobial ethylenimine polymer or co-polymer which comprises at least two different substituents according to claim 1.

12. A polymer or co-polymer according to claim 1 wherein a portion of the nitrogen atoms of the polymer or co-polymer backbone are substituted by two different substituents.

13. An antimicrobial ethylenimine polymer or co-polymer, wherein 10-99% of the nitrogen atoms of the ethylenimine polymer or co-polymer backbone are substituted according to claim 1 at least 1% of the N atoms of the ethylenimine polymer or co-polymer backbone are substituted by C₁₋₂₄ alkyl or alkyl substituted by —OH, —COOC₁₋₂₄ alkyl, —COOH or —COOM wherein M is as described above.

14. A method for protecting plastics, coatings, home or personal care formulations, industrial formulations or technical process against the action of microbes which comprises adding an effective amount of a polymer or copolymer of claim 1 to the formulation or process.

15. A method for protecting skin, mucosa and integumentary appendages against the action of microbes which comprises applying a preparation comprising an effective amount of a polymer or copolymer of claim 1.

16. A method for protecting paper, wood, leather or textile materials against the action of microbes comprising incorporating or applying an effective amount a polymer or copolymer of claim 1 or a composition comprising an effective amount a polymer or copolymer of claim 1.

17. A personal care preparation, oral hygiene formulation or washing and cleaning formulation comprising a polymer or copolymer of claim 1.

18. A composition comprising a polymer or copolymer of claim 1 and another natural or synthetic polymer.

19. A composition comprising more than one polymer or copolymer of claim 1.

20. A composition according to claim 18 which is a woven or non woven textile, paper product, coating composition or plastic article.

21. A method for cleaning and disinfecting hard surfaces which comprises applying a preparation comprising an effective amount of a polymer or copolymer of claim 1.

22. A method for preventing bio-fouling of an article comprising incorporating the antimicrobial ethylenimine polymers or co-polymers of claim 1 into the article or surface of the article or by applying the antimicrobial ethylenimine polymers or co-polymers to these surfaces either directly or as part of a coating or film.
